(19) United States
(12) Patent Application Publication
(10) Pub. No.: US 2006/0252985 A1
(43) Pub. Date: Nov. 9, 2006

(54) Male genital bib guard

(76) Inventor: Kim D. Bushey, Spruce, MI (US)

Correspondence Address: Kim D. Bushey, 2169 E. Swede Rd., Spruce, MI, 48762, US

(21) Appl. No.: 11/121,534

(22) Filed: May 4, 2005

(51) Int. Cl. A61F 5/00; A61F 6/02

(52) U.S. Cl. 600/38; 128/842

(57) ABSTRACT

A device related to a personal hygiene bib between a males penis and genitals. The disposable wearing article made of thin prophylactic or man made materials with (resilient) meaning stretch back quality or rubber band effect having no liquid absorbing quality factored therein. Usually in the form of a flexible thin elongated sheet type, of predetermined shapes and dimensions having various colors, writing or printed patterns, scented, or combinations of these elements and embodiments. The device, having a single perforated dotted shape push out circular hole or single cut out circle hole of designated size, usually towards the top of one end allowing the penis to enter through and slide up the base of the penis towards the pubic area therein front of the genitals forming a free standing or (staying) article with self retaining means, having no added attachments such as adhesive's or added on straps, buckles, belts, snaps.

### BACKGROUND OF THE INVENTION

A plethora of products are sold in the protection of or stopping the spreading of diseases. The present invention relates generally to a male genital bib guard, formed out of a disposable, usually a thin elongated flexible synthetic material. Which are readily and quickly easy to apply over the penis for a precautionary personal hygiene technique, against skin to skin contact with the female's sexual parts. Consequently, some prior art and conventional techniques of other inventions detour usage by cumbersome and discomfort devises such as genital caps, diapers, or to force the genitals into a condom device disenabling the freedom and comfort to the male's genital area. These items are timely and embarrassing to put on and restrict the circulation and perhaps losing the interest or perhaps change their minds and using nothing at all. So even if the said male genital bib guard was used as a sexual novelty device it still would be a precautionary attempt against catching or spreading some types of diseases.

### SUMMARY OF THE INVENTION

The present invention provides a personal hygiene method for a male genital bib guard. The fabrication comprising of man made materials or consisting of prophylactic, soft plastics, synthetic rubber or one, or more combination of all materials with (resilient) meaning stretch back quality or rubber band effect, without having any liquid absorbing quality factored therein said male genital bib guard, thus being configured into various different figured shapes to form usually a thin fabricated sheet types of predetermined dimensions and or of variable colors, lengths, width, thickness, writing or printed patterns, scented, and or different combinations of these elements and embodiments hereinabove. The present invention being of the comprised elements described hereinabove having said an elongated flexible body member of a predetermined dimension, within it having a single perforated dotted shape push out circular hole or single cut out circle hole, or a single perforated dotted push out slit or a single cut out slit of a predestinated size, usually towards the top of one end, of the said male genital bib guard. This allows the penis to enter through and slide up the base of the penis towards the pubic area therein front of the genitals, thus forming a free standing or (staying) article with self retaining means, having no added attachments such as adhesives or added on straps, buckles, belts, snaps, thus forming a disposable personal hygiene male genital bib guard barrier over the male's genital area. In addition to the present invention, because of materials used, that provides a (resilient) meaning stretch back quality or rubber band effect to remain into place on the portion of the penis, this enabling more comfort and freedom from restrictions or loss of circulation to the genitals. Other objects of the present invention is a manufactured version of the said male genital bib guard hereinabove which has tiny dotted perforated holes in a straight line from both ends but not connection in the center of the middle of the straight line, which leaves a remaining middle segment of a designated length not perforated, this predetermined line usually towards the top of one end thus enabling it to tear or separate forming two ribbon type tie strips. The lines leaving a segment in the middle portion can also be precut to form two ribbon tie strips. This version can be tied to the base of the penis for intensifying desirable pleasure. Other important aspect of the present invention said male genital bib guard described as hereinabove, is the capability of it being sterile and disposable thus making it a precautionary personal hygiene device against catching or spreading some viral infections and aiding in the protection against skin to skin contact from the female's sexual parts and also ministration fluids, sweat and anal bacteria, along with other sexual transmitted diseases like herpes, venereal wards, etc. Further objects, advantages, and features of the present invention will become apparent to those persons skilled in this particular area of technology and to other persons after having read the detailed description as set forth hereinbelow in conjunction with the accompanying drawings.

### BRIEF DESCRIPTION OF THE DRAWINGS OF THE INVENTION

FIG. 1 Illustrates a front view of the present invention having a single perforated dotted shape push out circular hole or single cut out circle hole of designated size within the said male genital bib guard.

FIG. 2 Illustrates a front view of the present invention having a single perforated dotted shape push out slit or cut out slit of designated size within the said male genital bib guard.

FIG. 3 Illustrates a front view of the present invention having tiny dotted perforated holes in a straight line from both ends but not connecting in the center of the middle of the straight line, which leaves a remaining middle segment of a designated length not perforated, enabling it to tear or separate forming two ribbon type tie strips with on the said male genital bib guard.

FIG. 4 Illustrates a front view of the present invention said male genital bib guard attached to a penis covering the male's genitals.

FIG. 5 Illustrates a sided view of the present invention said male genital bib guard attached to a penis towards the pubic area in front of the male's genitals.

FIG. 6 Illustrates a front view of the present invention said male genital bib guard having been tied to the penis with the two tear away ribbon type tie strips or pre cut ribbon type tie strip.

### DETAILED DESCRIPTION OF SOME PREFERRED EMBODIMENTS

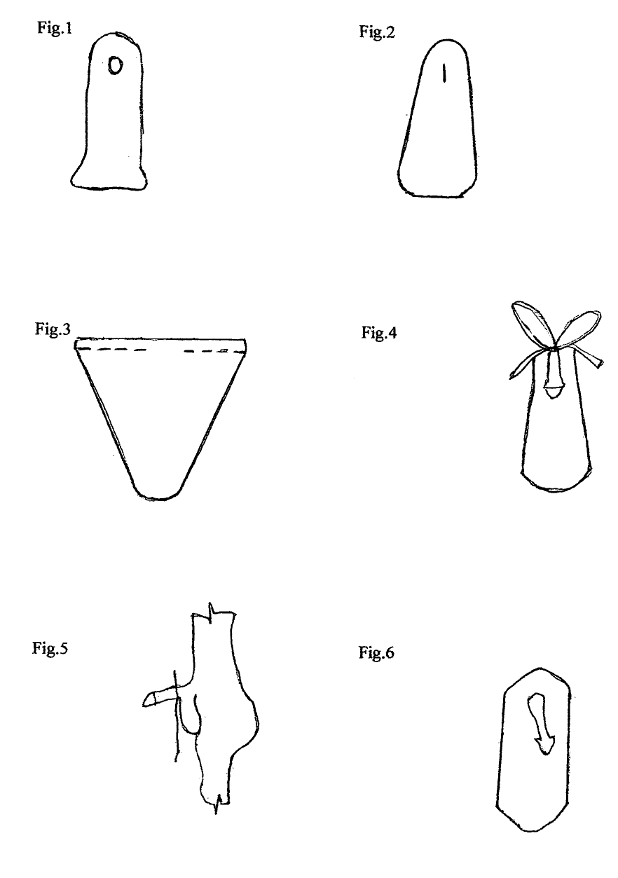

FIG. 1 shows a perspective view of the said male genital bib guard having a single perforated dotted shape push out circular hole or single cut out circle hole of designated size, being demonstrated in FIG. 4 on a male's penis. And FIG. 5 shows a side view projection on a male's penis of the FIG. 1 and FIG. 4

FIG. 2 is another perspective view of said male genital bib guard having a single perforated dotted shape push out slit or cut out slit of designated size, being demonstrated in FIG. 4 on a male's penis. And FIG. 5 shows a side view projection on a male's penis of the FIG. 2 and FIG. 4.

FIG. 3 shows the version of a said male genital bib guard having tiny dotted perforated holes in a straight line from both ends but not connecting in the center of the middle of the straight line, which leaves a remaining middle segment of designated length not perforated. Enabling it to tear or separate forming two ribbon type tie strips thus being tied around a male's penis in FIG. 6.

What I claim is my invention:

1. The present invention a said male genital bib guard being constructed of man made materials, consisting of prophylactic, soft plastics, syndic rubber or one or more combinations of able materials having (resilient) meaning stretch back quality or rubber band effect, without having no liquid absorbing quality factored therein, being constructed into various different forms, usually a thin fabricated sheet type, being of predetermined dimensions, shapes, various colors, lengths, width, thickness, writing or printed patterns, scented, and many figures of shapes and or combinations of these embodiments described hereinabove, said male genital bib guard being of the comprised elements and configurations described, having an elongated thin sheet type flexible body member with a single perforated dotted shape push out circular hole or single cut out circle hole of designated size within the said male bib guard, usually towards the top of one end, which allows the penis to enter through and slide up the base of the penis, towards the pubic area therein front of the genitals, thus forming a free standing or (staying) article with self retaining means, having no added attachments such as adhesive's, or added on straps, buckles, belts, snaps, thus forming a personal hygiene bib type barrier over the male's genital area.

2. Another presentation of the invention of claim 1, wherein said male genital bib guard, further comprised of a fabricated thin sheet type elongated flexible body member of predetermined dimensions and combinations of these elements and embodiments described hereinabove, with (resilient) meaning stretch back quality or rubber band effect, without having any liquid absorbing quality factored therein, thus having a single vertical or horizontal perforated dotted push out slit or single cut out slit of a designated size, usually towards the top of one end, which allows the penis to enter through and slide up the base of the penis, towards the pubic area therein front of the genitals, thus forming a free standing or (staying) article with self retaining means, having no added attachments such as adhesive's, or added on straps, buckles, belts, snaps, thus forming a disposable personal hygiene bib type barrier over the male's genital area.

3. Another presentation of the invention of claim 1, wherein said male genital bib guard further comprised of a fabricated thin sheet type elongated flexible body member of predetermined dimensions and or combinations of these elements and embodiments described hereinabove, with (resilient) meaning stretch back quality or rubber band effect, without having any liquid absorbing quality factored therein, thus having tiny dotted perforated holes in a straight line from both ends but not connecting in the center of the middle of the straight line, which leaves a remaining middle segment of a designated length not perforated, this predetermined line usually towards the top of one end, enabling it to tear or separate forming two ribbon tie strips, also the lines leaving a segment in the middle portions can also be precut to form two ribbon tie strips, this form of a said male genital bib guard is able to be tied around the base of the penis towards the pubic area therein front of the genitals, making a self retaining or (staying) article, thus forming a disposable personal hygiene bib type barrier over the male's genital area.

4. Another further presentation of the invention of claims 1, 2, 3, wherein said male genital bib guard being comprised of these elements and embodiments described hereinabove, having more then one or more combinations of different features made into one said male genital bib guard thus being constructed in varieties of a perforated dotted circular hole and slit, or cut out circle and slit, or precut and tear away ribbon tie strips or any mixed combinations of these formulas, usually assented towards the top of one end and bottom of one end.
